(19) United States
(12) Patent Application Publication
(10) Pub. No.: US 2015/0004711 A1
(43) Pub. Date: Jan. 1, 2015

(54) METHODS AND ASSAYS RELATING TO PROSTATE CANCER

(71) Applicant: The General Hospital Corporation, Boston, MA (US)

(72) Inventors: Anna Moore, Dracut, MA (US); Zdravka Medarova, Methuen, MA (US)

(73) Assignee: The General Hospital Corporation, Boston, MA (US)

(21) Appl. No.: 14/378,146

(22) PCT Filed: Feb. 21, 2013
(86) PCT No.: PCT/US13/27022

§ 371 Date: Aug. 12, 2014

Related U.S. Application Data
(60) Provisional application No. 61/602,223, filed on Feb. 23, 2012.

(51) Int. Cl. G01N 33/20

(52) U.S. Cl. CPC G01N 33/20; G01N 2800/52
USPC 436/81

(57) ABSTRACT

The technology described herein relates to determining the zinc score in a sample obtained from a subject, e.g. for treating a subject for prostate cancer.

### CROSS-REFERENCE TO RELATED APPLICATIONS

This application claims benefit under 35 U.S.C. §119(e) of U.S. Provisional Application No. 61/602,223 filed Feb. 23, 2012, the contents of which are incorporated herein by reference in their entirety.

### GOVERNMENT SUPPORT

This invention was made in part with U.S. Government support from grant R00CA129070 from the National Cancer Institute and grant GM-65519 from the National Institute of General Medical Sciences. The U.S. Government has certain rights in this invention.

### FIELD OF THE INVENTION

The invention relates to assays for determining the likelihood of a subject having prostate cancer based upon a quantitative zinc score.

### BACKGROUND OF THE INVENTION

As a ubiquitous and indispensible micronutrient in the human body, zinc ions (Zn²⁺) are present in every mammalian cell and play a pivotal role in a broad range of fundamental physiological functions. The forms of biological zinc can be divided into two categories: tightly bound zinc, which serves as structural and catalytic components of metalloprotein scaffolds, and mobile zinc, which exists in certain mammalian organs, including the brain, retina, pancreas, and prostate, and functions as an essential molecular signaling agent. The homeostasis of Zn²⁺, the process that controls a balanced level of the ion in all tissues, is strictly regulated under physiological conditions. When this process breaks down, as in diseases such as prostate cancer, there is a significant disruption of the proper, physiologically controlled levels of extra- and intracellular mobile [Zn²⁺]. The ability to measure and accurately quantify mobile zinc ions in these organs offers a potentially powerful method for early diagnosis of such zinc-related diseases, such as prostate cancer.

The healthy prostate contains high concentrations of mobile zinc, which decrease significantly during the development of prostate cancer, even at an early stage. Thus reduced zinc levels are a biochemical hallmark of prostate cancer development. The importance of diagnosing prostate cancer early is indisputable. Prostate cancer is the second leading cause of cancer death in men, exceeded only by lung cancer. According to the American Cancer Society, it accounts for about 13 percent of male cancer-related deaths. In its early stages, when it is still curable, prostate cancer causes no symptoms. Notably, the 5-year disease-specific survival rates for localized cancer are 100%. By contrast, metastatic prostate cancer is not curable and has an overall 5-year survival of just 33%. Life expectancy can be as low as 13 months, even in the presence of androgen-deprivation therapy (see Mannuel, H. D.; Hussain, A. Clin. Genitourin. Cancer 2006, 5, 43-9). Consequently, the ability to diagnose prostate cancer early, before it has spread beyond the confines of the organ, could offer the only possibility of a cure to patients at risk for aggressive disease. Indeed, with the advent of routine testing for serum prostate specific antigen (PSA), the 5-year cancer-specific survival rates increased from approximately 70% in the early 1980s to over 90% just a decade later. The significance of early diagnosis and intervention is especially pronounced when considering younger men with a longer life expectancy. A recent study found that, in men under the age of 50, disease-specific survival at 16 years was 73%, whereas of the men treated with radical prostatectomy, 94% were alive at 21 years of observation. Men under 50 more commonly had organ-confined disease and a pathologic Gleason score ≦6, implying that early diagnosis of disease is associated with more favorable outcomes and that population-based screening at younger ages could potentially lead to improved survival (3).

However, there is thus an ongoing need for assays for determining the likelihood of a subject having prostate cancer. Such assays can be of use in clinical applications including diagnosing and staging prostate cancer, as well as differentiating prostate cancer from conditions such as benign prostatic hyperplasia and prostatitis. The field suffers from a lack of reliable, clinically applicable diagnostic and staging tools for prostate cancer. The current clinical diagnosis and staging of a prostate cancer relies on four core parameters: digital rectal examination (DRE), serum prostate-specific antigen (PSA), biopsy, and imaging (4). Among them, the PSA test represents an important initial screening tool. Although many men with prostate cancer have an elevated PSA concentration (greater than 4.0 ng/mL), a high level does not necessarily mean that there is cancer. A false positive rate as high as 70% has been reported (5). Conversely, 15% of men with prostate cancer will not have an elevated PSA measurement. In fact, close to 7% of men with a PSA of 0.5 ng/mL or lower will have prostate cancer and, of those, 12% will have high-grade disease (6). Improvements based on the PSA test, such as the measurement of PSA velocity and free PSA, are also used clinically. However, neither PSA velocity (7) nor free PSA have consistently been shown to enhance the specificity of prostate cancer detection over PSA level alone (8).

This means that many men will either undergo unnecessary invasive and emotionally taxing treatment or remain undiagnosed. Therefore, a clinically applicable assay directed to a truly predictive biomarker is urgently needed in order to aid in accurately diagnosing subjects and stratifying the risk faced by those subjects. Such an assay would resolve the current diagnostic dilemma between watchful waiting and aggressive treatment.

### SUMMARY OF THE INVENTION

Described herein are methods and assays directed to determining the likelihood of a subject having prostate cancer and treating prostate cancer. The assays described herein relate to zinc as a prostate biomarker, which, despite having been known to the scientific community for over 50 years, has not fulfilled its promise in a clinical setting; owing to the absence of appropriate clinically relevant tools. In some aspects, the assays described herein provide a simple clinical assay for determining a clinically relevant zinc score. The methods and assays described herein which relate to determining a zinc score in a sample obtained from a subject are based upon the inventors' discovery that a zinc score determined by precise quantitative determination of the zinc content in a prostate secretion urine sample obtained from the subject followed by normalizing the zinc content in the sample to a control substance, provides a clinically relevant quantitative measure of the likelihood of a subject having prostate cancer. Further aspects of the methods and assays described herein are based upon the inventors' discovery of zinc score thresholds that indicate increases in the likelihood of a subject having prostate cancer, a high-risk form of prostate cancer, or a prostate cancer which is in need of treatment.

In one aspect, the invention described herein relates to a method and/or assay for determining the likelihood of a subject having prostate cancer, comprising: (i) determining the level of zinc and a control substance in an expressed prostate secretion urine sample obtained from the subject; (ii) determining a zinc score, wherein the zinc score is equal to the concentration of zinc multiplied by the control substance concentration; wherein the subject is at risk of prostate cancer if the score is 85% or less of the average zinc score for a population of healthy adult males.

In some embodiments, the level of zinc is determined using ZPP1. In some embodiments, the level of zinc is determined using a titration assay. In some embodiments, the control substance comprises creatinine. In some embodiments, the zinc score is determined by multiplying the level of zinc in uM by the level of creatinine in ug/dL.

In some embodiments, the subject is at risk of having a high-risk prostate cancer if the zinc score is less than 55% of the average zinc score for a population of healthy adult males.

In some embodiments, the zinc score of the subject is determined for a sample obtained at a first date and further comprising determining the zinc score in at least one additional sample obtained at a later date; wherein a decrease in the zinc score over time indicates the subject is at risk of having or developing prostate cancer.

In some embodiments, if the zinc score is less than 55% of the average zinc score for a population of healthy adult males, the subject is recommended to be treated with a treatment selected from the group consisting of: radial prostatectomy; chemotherapy; temozolomide; hormone therapy; radiation therapy; external beam radiation therapy; brachytherapy; cryosurgery; high-intensity focused ultrasound; and surgery. In some embodiments, if the score is greater than 85% of the average zinc score for a population of healthy adult males, the subject is recommended to not receive treatment for prostate cancer. In some embodiments, if the score is less than 85% and more than 55% of the average zinc score for a population of healthy adult males, the subject is recommended to undergo further testing or monitoring for prostate cancer.

In one aspect, the invention described herein is directed to a method and/or assay for determining the likelihood of a subject having prostate cancer, comprising: (i) determining the level of zinc and a control substance in an expressed prostate secretion urine sample obtained from the subject; (ii) determining a zinc score, wherein the zinc score is equal to the concentration of zinc multiplied by the control substance concentration; wherein the subject has a increased likelihood of having prostate cancer if the score is or is equivalent to a zinc-creatinine score of 7 or less.

In some embodiments, the level of zinc is determined using ZPP1. In some embodiments, the level of zinc is determined using a titration assay. In some embodiments, the control substance comprises creatinine. In some embodiments, the zinc score is determined by multiplying the level of zinc in uM by the level of creatinine in ug/dL.

In some embodiments, the subject is at risk of having a high-risk prostate cancer if the zinc score is or is equivalent to a zinc-creatinine score of less than 4. In some embodiments, the zinc score of the subject is determined for a sample obtained at a first date and further comprising determining the zinc score in at least one additional sample obtained at a later date; wherein a decrease in the zinc score over time indicates the subject is at risk of having or developing prostate cancer.

In some embodiments, if the zinc score is or is equivalent to a zinc-creatinine score of less than 4, the subject is recommended to be treated with a treatment selected from the group consisting of: radial prostatectomy; chemotherapy; temozolomide; hormone therapy; radiation therapy; external beam radiation therapy; brachytherapy; cryosurgery; high-intensity focused ultrasound; and surgery. In some embodiments, if the score is or is equivalent to a zinc-creatinine score of 7 or more, the subject is recommended to not receive treatment for prostate cancer. In some embodiments, if the zinc score is or is equivalent to a zinc-creatinine score of more than 4 and less than 7, the subject is recommended to undergo further testing or monitoring for prostate cancer.

In one aspect, the invention described herein is directed to a system for obtaining data from at least one expressed prostate secretion urine sample obtained from at least one subject, the system comprising: (i) a determination module configured to receive the at least one expressed prostate secretion urine sample and perform at least one analysis on the at least one expressed prostate secretion urine sample to determine a zinc score; (ii) a storage device configured to store data output from said determination module; and (iii) a display module for displaying a content based in part on the data output from said determination module, wherein the content comprises a signal indicative of the zinc score.

In some embodiments, the system further comprises a means of inputting a value for the level of one or more of zinc or a control substance determined to be in the expressed prostate secretion urine sample. In some embodiments, the content displayed on said display module further comprises a signal indicative of the subject being at risk of having or developing prostate cancer if the zinc score is determined to be less than 85% of the average zinc score in a population of healthy adult males. In some embodiments, the content displayed on said display module further comprises a signal indicative of the subject being at risk of having or developing prostate cancer if the zinc score is determined to be or be equivalent to a zinc-creatinine score of less than 7. In some embodiments, the content displayed on said display module further comprises a signal indicative of the subject being recommended to further testing or monitoring for prostate cancer. In some embodiments, the content displayed on said display module further comprises a signal indicative of the subject being at risk of having or developing prostate cancer if the zinc score is determined to be less than 85% of the average zinc score in a population of healthy adult males. In some embodiments, the content displayed on said display module further comprises a signal indicative of the subject being at risk of having or developing prostate cancer if the zinc score is determined to be or be equivalent to a zinc-creatinine score of less than 4. In some embodiments, the content displayed on said display module further comprises a signal indicative of the subject being recommended to receive treatment for prostate cancer.

### BRIEF DESCRIPTION OF THE DRAWINGS

FIGS. 1A-1B depict zinc measurement by ZPP1 titration in serial dilutions of an EPS-urine sample from a non-cancer patient. FIG. 1A: Representative titration plot. FIG. 1B: Correlation between sample dilution and the zinc concentration measured using the ZPP1 assay over the range of tested dilutions. There was a high linear correlation (R2=0.998) between sample dilution and the zinc concentration measured using the ZPP1 assay.

FIG. 2 depicts quantification of zinc concentration by ZPP1 titration in EPS urine from a pool of non-cancer and cancer patients. The measured zinc concentration was 3.5-fold higher in the non-cancer group.

FIGS. 3A-3B depict data analysis. This figure illustrates the posterior probability of disease category as functions of zinc score (FIG. 3A) and PSA (FIG. 3B). The quantities on the horizontal axes are assumed to be normally distributed given the category (i.e., Benign, Low-Risk, or High-Risk). The priors for the normal means and standard deviations are constant and reciprocal, respectively. The prior for the category probabilities is the proportions of the data in each category. The colored dots are the data values in each category. The colored bars indicate which category has the highest probability as function of the zinc score or PSA. Darker grey=benign, lighter grey=low risk, black=high risk.

FIG. 4 is a diagram of an embodiment of a system for performing an assay for determining the zinc score for an EPSU sample obtained from a subject.

FIG. 5 is a diagram of an embodiment of a comparison module as described herein.

FIG. 6 is a diagram of an embodiment of an operating system and applications for a computing system as described herein.

### DETAILED DESCRIPTION OF THE INVENTION

Described herein are methods, assays, and systems relating to determining the likelihood of a subject having prostate cancer. The assays described herein rely upon the inventors' demonstration of a clinically meaningfully assay directed to determining a zinc score and the correlation of the results of such assays with a subject's risk of having and/or developing prostate cancer.

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); The ELISA guidebook (Methods in molecular biology 149) by Crowther J. R. (2000). Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8).

Unless otherwise stated, the present invention was performed using standard procedures, as described, for example in U.S. Pat. Nos. 4,965,343, and 5,849,954; Davis et al., Basic Methods in Molecular Biology, Elsevier Science Publishing, Inc., New York, USA (1995); Current Protocols in Cell Biology (CPCB) (Juan S. Bonifacino et. al. ed., John Wiley and Sons, Inc.); which are all incorporated by reference herein in their entireties.

The terms “decrease,” “reduce,” “reduced”, and “reduction” are all used herein generally to mean a decrease by a statistically significant amount relative to a reference. However, for avoidance of doubt, “reduce,” “reduction”, or “decrease” typically means a decrease by at least 10% as compared to the absence of a given treatment and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the absence of a given treatment, or any decrease between 10-99% as compared to the absence of a given treatment.

The terms “increased”, “increase”, or “enhance” are all used herein to generally mean an increase by a statically significant amount; for the avoidance of any doubt, the terms “increased”, “increase”, or “enhance” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein in the context of expression, the terms “treat,” “treatment,” and the like, as used in the context of the therapeutic methods described herein, refer to a decrease in severity, indicators, symptoms, and/or markers of prostate cancer as described herein. In the context of the present invention insofar as it relates to any of the conditions recited herein, the terms “treat,” “treatment,” and the like mean to relieve, alleviate, ameliorate, inhibit, slow down, reverse, or stop the progression, aggravation, deterioration, anticipated progression or severity of at least one symptom or complication associated with prostate cancer. In one embodiment, the symptoms of prostate cancer are alleviated by at least 10%, at least 20%, at least 30%, at least 40%, or at least 50%.

As used herein, the term “administer” refers to the placement of a composition into a subject by a method or route which results in delivery of at least part of the administered composition to a desired site such that the desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, topical (including buccal and sublingual), intracranial, and intracerebral administration.

In some embodiments, a “subject” as used herein can be a male human or male animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf. Patient or subject includes any subset of the foregoing, e.g., all of the above. In certain embodiments, the subject is a mammal, e.g., a primate, e.g., a human.

Preferably, the subject is a mammal. The mammal can be a human, non-human primate, mouse, rat, dog, cat, horse, or cow, but is not limited to these examples. Mammals other than humans can be advantageously used as subjects that represent animal models of prostate cancer. In addition, the methods and assays described herein can be used to treat domesticated animals and/or pets. A subject can be one who has been previously diagnosed with or identified as suffering from or having prostate cancer or one or more complications related to prostate cancer, and optionally, but need not have already undergone treatment for prostate cancer or the one or more complications related to prostate cancer. Alternatively, a subject can also be one who has not been previously diagnosed as having prostate cancer or one or more complications related to prostate cancer. For example, a subject can be one who exhibits one or more risk factors for prostate cancer or one or more complications related to prostate cancer or a subject who does not exhibit prostate cancer risk factors.

As used herein, a “portion” refers to a part or fraction of a whole, e.g. a part or fraction of a total sample volume.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

Other than in the operating examples, or where otherwise indicated, all numbers expressing quantities of ingredients or reaction conditions used herein should be understood as modified in all instances by the term “about.” The term “about” when used in connection with percentages can mean±1%.

Other terms are defined herein within the description of the various aspects of the invention.

In some embodiments, the methods and assays described herein relate to determining the level of zinc and a control substance in an expressed prostate secretion urine sample obtained from a subject. As used herein “expressed prostate secretion urine sample” and “EPSU sample”, which are used interchangeably herein, refer to a urine sample obtained from a male subject after that subject has undergone a procedure to express secretions and/or fluids from the prostate into the urethra. In some embodiments, the prostate secretions and/or fluids are expressed from the prostate into the urethra via prostate massage. In some embodiments, the prostate massage comprises 3 strokes with a digit to each side of the median sulcus of the prostate during a digital rectal examination. The expressed prostate secretion urine sample can be collected at any time after the prostate secretion is expressed into the urethra provided that the urine sample comprises the first release of urine from the urethra following the expression of the prostate secretion. In some embodiments, the expressed prostate secretion urine sample can be collected within 1 hour of the prostate secretion being expressed into the urethra.

In some embodiments, the EPSU sample can be freshly collected. In some embodiments, the EPSU sample can be stored prior to being used in the methods and assays described herein. In some embodiments, the EPSU sample is stored on ice after collection. In some embodiments, the EPSU sample is stored at 4° C. after collection. In some embodiments, the EPSU sample is frozen after collection. A frozen sample can be thawed before being used in the methods and assays described herein. In some embodiments, the EPSU sample is a clarified fluid sample, for example, by centrifugation. In some embodiments, the sample is clarified by low-speed centrifugation (e.g. 3,000×g or less) and collection of the supernatant comprising the clarified fluid sample. In some embodiments, the EPSU sample can be centrifuged at 2500 rpm for 5 min. In some embodiments, the EPSU sample can be diluted. In some embodiments, the EPSU sample can be diluted in a buffer, e.g. HEPES/KCl buffer. In some embodiments, the EPSU sample used in the methods described herein is an untreated sample. As used herein, “untreated sample” refers to a sample that has not had any prior sample pre-treatment except for dilution and/or suspension in a solution.

In some embodiments, the EPSU sample used in the methods described herein is a treated sample. Exemplary methods for treating or processing a sample include, but are not limited to, centrifugation, filtration, sonication, homogenization, heating, freezing and thawing, contacting with a preservative (e.g. protease inhibitor) and any combination thereof. In some embodiments, the sample can be treated with a chemical and/or biological reagent. Chemical and/or biological reagents can be employed to protect and/or maintain the stability of the sample during processing and/or storage. The skilled artisan is well aware of methods and processes for processing, preservation, or treatment of biological and/or urine samples.

In some embodiments, the methods, systems, and assays described herein relate to determining whether a subject has an increased likelihood of having prostate cancer. As used herein, “prostate cancer” refers to any cancer of the prostate gland in which cells of the prostate mutate and begin to multiply out of control. The term “prostate cancer” includes early stage, localized cancer of the prostate gland; later stage, locally advanced cancer of the prostate gland; and later stage metastatic cancer of the prostate gland (in which the cancer cells spread (metastasize) from the prostate to other parts of the body, especially the bones and lymph nodes). Prostate cancer can include adenocarcinoma, leiomyosarcoma, rhabdomyosarcoma, intraepithelial neoplasia, or sarcoma. Prostate cancers can be encapsulated or metastatic. Symptoms, signs and markers of prostate cancer can include, but are not limited to, frequent urination, urgent urination, nocturia, difficulty in starting or stopping the urine stream, inability to urinate standing up, painful urination or ejaculation, enlarged prostate, blood in the mine, blood in the semen, impotence, bone pain (particularly in the hips, ribs, or lower back), swelling the legs, decreased force in the urine stream, an interrupted urine stream, and loss of bladder control.

In some embodiments, the assays described herein relate to determining whether a subject has an increased likelihood of having a high-risk prostate cancer. As used herein, the term “high-risk prostate cancer” refers to prostate cancer that has metastasized in a subject or is likely to metastasize in a subject, including malignant tumors with high potential for metastasis (prostate cancer that is considered to be aggressive).

In some embodiments, the assays described herein comprise determining the level of zinc in the ERSU sample obtained from a subject. In some embodiments, the level of free, mobile, and/or readily available zinc in an EPSU sample is determined. In some embodiments, the level of free, mobile, and/or readily available zinc in the non-cellular fraction of an EPSU sample is determined. In some embodiments, an EPSU sample is centrifuged to pellet the cells present in the sample and the supernatant is used to determine the level zinc in the EPSU sample. In some embodiments, the level of intracellular or bound zinc in an EPSU sample is not determined.

Methods of determining the level of zinc in a sample include, but are not limited to optical detection of a signal in a colorimetric, absorptionmetric, or fluoremetric assay. A zinc-sensitive chromophore or fluorophore, that is, a moiety that will absorb or emit different amounts or wavelengths of light when bound to zinc, is contacted with a sample or portion thereof comprising zinc and the amount of light absorbed or emitted is compared to a control sample, control value, or the value prior to contacting the zinc-sensitive moiety with a sample or portion thereof. Zinc-sensitive chromophores and fluorophores are well known in the art. Non limiting examples of zinc-sensitive chromophores and fluorophores include, quinoline, RAPTA, ethylene diamine tetra acetic acid, pyridine, TPEN, P.A.R., 8-hydroxy quinoline, Eriochrome black, Alloxan tetrahydrate, Arsenazo III, Calconcarboxylic acid, Calmagite, Chromeazuro 1 1,5-Diphenylcarbazide, Diphenylcarbazone, Dithizone, Eriochrome Black, Hydroxynaphthol blue, Methylthymol Blue, 1-(2Pyridylazo)-2-naphthol, Pyrocatechol 5-Sulfosalicylic acid dehydrate, Tiron, Zincon and 2-(5-Bromo-2pyridylazo)-5-(N-propyl-N-sulfopropylamino)phenol (5-BrPAPS), 4-(2-pyridylazo) resorcinol, fluorescein, rhodamine, allexa, and dansylamide. Zinc-sensitive chromophores and fluorophores are commercially available, for example, dithizone (Cat No. 194832 Sigma-Aldrich; St. Louis, Mo.) and zincon (Cat No. 33826 Sigma-Aldrich; St. Louis, Mo.).

In some embodiments, the zinc-sensitive moiety can be mixed with the EPSU sample or a portion thereof. In some embodiments, the zinc-sensitive moiety is attached to the surface of a solid substrate.

In some embodiments, the zinc-sensitive moiety can comprise a zinc sensor compound, that is, any compound that contains an optical reporter having two or more recognition units where each of the recognition units is capable of associating with at least one zinc ion. As used herein, a “zinc ion” is any one of the ions of zinc that are known to exist, including, but not limited to being, Zn²⁺. The optical reporter can be any component of the zinc sensor compound that has the ability to cause the molecule to provide an optical response, e.g. a component that causes the molecule to be fluorescent. Suitable optical reporters include fluorophore, such as fluorescein or its analogs, such as 2′,7′-dichlorofluorescein and 2′,7′-difluorofluorescein. In certain instances, the fluorophore is more fluorescent when two or more zinc ions are associated with the recognition units than the fluorophore is when no zinc ions are associated with the recognition units or when one zinc ion is associated with the recognition units. A “recognition unit” means anything that is capable of associating with at least one zinc ion. Therefore, each one of “zinc sensor,” “zinc-binding unit” and “analyte binding unit” is an exemplary “recognition unit,” but it is to be understood that “recognition unit” is not limited thereto those examples and therefore is to be construed more broadly than each one of those examples. Other recognition units are contemplated and will be readily recognized by the skilled artisan. Two or more recognition units should be present on the zinc sensor compound. In some embodiments, the compound may contain exactly two recognition units. The recognition units may have the same or different structures. Dipicolylamine and analogs of dipicolylamine, such as 1-(pyrazin-2-yl)-N-(pyridin-2-ylmethyl)methanamine, are examples of suitable recognition units.

Non-limiting examples of zinc sensor compounds and methods of producing them are described in U.S. Pat. No. 7,160,732 and International Patent Publication PCT/US2010/045245 which are incorporated by reference herein in their entirety. In a preferred embodiment, the zinc sensor compound is the following compound, also referred to as ZPP1:

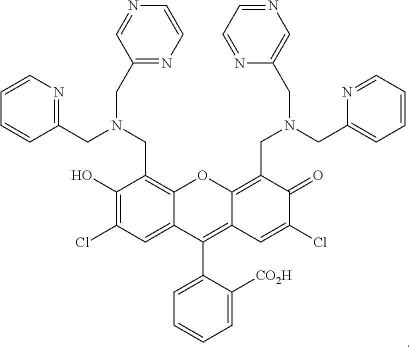 [SMILES: O=C(O)c1ccccc1-c1c2cc(Cl)c(=O)c(CN(Cc3ccccn3)Cc3cnccn3)c-2oc2c(CN(Cc3ccccn3)Cc3cnccn3)c(O)c(Cl)cc12]

For ZPP1, the “recognition unit” is 1-(pyrazin-2-yl)-N-(pyridine-2-ylmethyl)methanamine, and the “optical reporter” is 2′,7′-dichlorofluorescein.

In some embodiments, the level of zinc in an EPSU sample can be determined using a titration assay. As used herein, a “titration assay” refers to determining the result obtained in a series of assay reactions in which one of the components of the reaction is increased in a stepwise manner. In some embodiments, the concentration of the zinc-sensitive moiety and/or zinc sensor compound is increased while the concentration of the EPSU sample is the same. In some embodiments, the level of zinc in an EPSU sample can be determined using a titration assay and a zinc sensor compound. In some embodiments, the level of zinc in an EPSU sample can be determined using a titration assay and ZPP1. In some embodiments, the level of zinc in an EPSU sample can be determined as follows. EPSU samples can be centrifuged at 2500 rpm for 5 min and diluted (1:4) in HEPES/KCl buffer (25 mmol/L HEPES and 100 mmol/L KCl; pH 7.0). For fluorescence analysis of zinc, 0.1 ml diluted EPSU samples can be added to 96-well plates. ZPP1 can be titrated into the sample to achieve stepwise increments in ZPP1 concentration. At each step, the fluorescence can be measured (excitation, 505 nm; emission, 532 nm) using a SpectraMax M2 fluorescence spectrophotometer (Molecular Devices). For each measurement, the fluorescence of buffer containing and equivalent amount of ZPP1 alone can be subtracted from that of the sample. Because of the chemical properties of ZPP1, the titration curve gives a maximum when its concentration is exactly half that of the mobile zinc in the test solution. The zinc concentration can then be calculated according to the formula:

[Zn²⁺]=2[ZPP1]ₘₐₓ

Using this method, the measurements can be linear over a range of 0.02-5 mM [Zn²⁺].

In some embodiments, the assays described herein comprise determining the level of a control substance in the EPSU sample obtained from a subject. As used herein, a “control substance” is a substance which can be found at an approximately constant concentration in male urine. Non-limiting examples of control substances include, creatinine and urea. Methods of detecting control substances are well known to those of ordinary skill in the art. By way of non-limiting example, in one embodiment, the control substance is creatinine. While urinary creatinine levels have been reported to vary depending on, for example, time of sampling, age, sex, race/ethnicity. BMI, and fat-free mass (Barr et al. Environmental Health Perspectives 2005 113:192-200; Greenblatt et al. J Clin Pharmacol 1976 16:321-8; Boeniger et al. American industrial Hygiene Association Journal 1993 54:615-627; which are incorporated by reference herein in their entirety), the inventors have demonstrated herein the surprising finding that urinary creatinine levels can be used to determine a clinically meaningful zinc score according to the methods and assays described herein. One of ordinary skill in the art is familiar with spectroscopic methods of determining the concentration of creatinine in a sample. By way of non-limiting example, the level of creatinine can be determined by a colorimetric assay using picric acid, which reacts with creatinine. Kits for determining the creatinine level in a sample are commercially available, e.g. Cat. No. 500701 from Cayman (Ann Arbor, Mich.) or Cat No. ab65340 from AbCam (Cambridge, Mass.). In some embodiments, the results of an assay to determine the level of creatinine or another control substance are detected using a spectrophotometer, e.g. the SpectraMax M2 fluorescence spectrophotometer (Molecular Devices). In some embodiments, the results of an assay to determine the level of creatinine or another control substance are detected using an automated device, e.g. the SpectraMax M2 fluorescence spectrophotometer (Molecular Devices).

In some embodiments, the assays described herein relate to determining the level of zinc and a control substance in an EPSU sample obtained from a subject and determining the zinc score. As used herein, “zinc score” refers to the numerical score obtained by multiplying the concentration of zinc in the EPS sample by the concentration of the control substance (e.g. creatinine) in the sample. The zinc score thus reflects an adjusted level of zinc in the EPSU sample to control for what fraction of the tool EPSU volume is from the expressed prostate secretion as opposed to the fraction comprising urine.

In some embodiments, the zinc score is a zinc-creatine score. As used herein, the term “zinc-creatinine score” refers to a numerical score calculated by multiplying the level of zinc determined to be in the EPSU sample (in uM units) by the level of creatinine determined to be in the EPSU sample (in ug/dL units).

In some embodiments, the zinc score determined from the EPSU sample obtained from a subject is compared to the average zinc score from a population of healthy adult males. In some embodiments, the zinc score determined from the EPSU sample obtained from a subject is of the same “type” of zinc score as the average zinc score from a population of healthy adult males. As used herein, two zinc scores are of the same type if they have been determined using the same assay for zinc, the same units of zinc, the same control substance, the same assay for the control substance, and the same units of the control substance. In some embodiments, zinc scores of different types can be compared by determining a conversion factor such that a zinc score of a first type can be converted to a score equivalent to a zinc score of a second type. By way of non-limiting example, if a first and second zinc score type vary in that the first zinc score type uses the concentration of zinc in mM and the second zinc score type uses the concentration of zinc in uM, the first zinc score type can be converted to a zinc score type equivalent to the second zinc score type by multiplying the first zinc score by 1000. In a further non-limiting example, if a first zinc score type and second zinc score type differ in that the first zinc score type is calculated using a first control substance which is known to be present at a concentration 10-fold higher than a second control substance which is used to calculate a second zinc score type, then a zinc score of the first zinc score type can be converted into a zinc score type equivalent to the second zinc score type by dividing the first zinc score by 10. In some embodiments, in order to establish a conversion score for two types of zinc scores, both types of zinc scores can be determined for a number of control samples and a conversion factor can be mathematically derived for each pair of zinc score types. In some embodiments, wherein in order to establish a conversion score for two types of zinc scores, both types of zinc scores are determined for a number of control samples and a conversion factor is mathematically derived for each pair of zinc score types, the average of the conversion factors obtained for the control samples can be used as a conversion factor. How to convert zinc scores determined by different means will be readily understood by one of ordinary skill in the art.

In some embodiments, when the zinc score determined for a subject is compared to the average zinc score for a population of healthy adult males, the zinc scores are of the same type. In some embodiments, when the zinc score determined for a subject is compared to the average zinc score for a population of healthy adult males, the zinc scores are of different types. In some embodiments, when the zinc score determined for a subject is compared to the average zinc score for a population of healthy adult males, the zinc scores are of different types and a conversion factor can be known or determined and used to convert one of the zinc score types to a second type of zinc score.

In some embodiments, the methods and assays described herein relate to determining if a zinc score is or is equivalent to a zinc-creatinine score of a given value. As used herein, “is equivalent to a zinc-creatinine score” refers to a zinc score which is not a zinc-creatinine score as defined herein but which, when converted to a zinc-creatinine score will be a score within the given range. Conversion of zinc scores from one type to a second type is described above herein.

In some embodiments, a subject is determined to be at risk of having prostate cancer if the zinc score is determined to be 85% or less of the average zinc score for a population of healthy adult males, e.g., if the zinc score is 85% or less, 70% or less, 50% or less, 30% or less, or 20% or less of the average zinc score for a population of healthy adult males. In some embodiments, a subject is determined to be at risk of having a high-risk prostate cancer if the zinc score is 55% or less of the average zinc score for a population of healthy adult males, e.g. if the zinc score is 45% or less. 30% or less, 20% or less, 15% or less, or 10% or less of the average zinc score for a population of healthy adult males. In some embodiments, a subject is determined to be at risk of having prostate cancer if the zinc score is determined to be 30% or less of the average zinc score for a population of healthy adult males and a subject is determined to be at risk of having a high-risk prostate cancer if the zinc score is 20% or less of the average zinc score for a population of healthy adult males. In some embodiments, a subject is determined to be at risk of having prostate cancer if the zinc score is, or is equivalent to a zinc-creatinine score of 7 or less. In some embodiments, a subject is determined to be at risk of having a high-risk prostate cancer if the zinc score is or is equivalent to a zinc-creatinine score of less than 4. As used herein “at risk of having” refers to at least a 2-fold greater likelihood of having a particular condition as compared to a male that did not meet the threshold score, e.g. a 2-fold, or 2.5-fold, or 3-fold, or 4-fold, or greater risk.

As used herein, a “healthy adult male” can be one who does not display any markers, signs, or symptoms of prostate cancer and who is not at risk of having prostate cancer. By way of non-limiting example, a healthy adult male will have no urination problems. In some embodiments, the population of healthy adult males can include males with similar demographic characteristics as the subject, e.g. similar age, similar ethnic background, similar diets, etc.

In some embodiments of the assays described herein, the zinc score of the subject is determined for a sample obtained at a first date and at least one additional sample obtained at a later date. In some embodiments, a decrease in the zinc score over time indicates an increased risk of the subject having prostate cancer. In some embodiments, a decrease in the zinc score over time indicates the subject is at risk of having prostate cancer. In some embodiments, a decrease in the zinc score of at least 10% over time indicates the subject is at risk of having prostate cancer, e.g. a decrease of at least 10%, 15%, 20%, 25%, 30%, 50%, 75% or more indicates the subject is at risk of having prostate cancer. In some embodiments, a decrease in the zinc score over time from a zinc score which is or is equivalent to a zinc-creatine score greater than 7 to a zinc score which is or is equivalent to a zinc-creatine score less than 7 indicates the subject is at risk of having prostate cancer. In some embodiments, a decrease in the zinc score over time from a zinc score which is or is equivalent to a zinc-creatine score greater than 7 to a zinc score which is or is equivalent to a zinc-creatine score less than 4 indicates the subject is at risk of having a high-risk prostate cancer. In some embodiments, a decrease in the zinc score over time from a zinc score which is or is equivalent to a zinc-creatine score less than 7 but greater than 4 to a zinc score which is or is equivalent to a zinc-creatine score less than 4 indicates the subject is at risk of having a high-risk prostate cancer.

In some embodiments, a decrease in the zinc score over time from a zinc score which is greater than 85% of the average zinc score of a population of healthy adult males to a zinc score which is less than 85% of the average zinc score of a population of healthy adult males indicates the subject is at risk of having prostate cancer. In some embodiments, a decrease in the zinc score over time from a zinc score which is greater than 85% of the average zinc score of a population of healthy adult males to a zinc score which is less than 55% of the average zinc score of a population of healthy adult males indicates the subject is at risk of having a high-risk prostate cancer. In some embodiments, a decrease in the zinc score over time from a zinc score which is less than 85% but more than 55% of the average zinc score of a population of healthy adult males to a zinc score which is less than 55% of the average zinc score of a population of healthy adult males indicates the subject is at risk of having a high-risk prostate cancer.

In some embodiments, if the zinc score of the subject is determined to be less than 55% of the average zinc score of a population, the subject is recommended to receive and/or be administered a treatment for prostate cancer, e.g. if the zinc score of the subject is determined to be 55% or less, or 45% or less, or 35% or less, or 25% or less, or 20% or less, or 15% or less, or 10% or less than of the average zinc score of a population, the subject is recommended to receive and/or be administered a treatment for prostate cancer. In some embodiments, if the zinc score of the subject is determined to be less than 15% of the average zinc score of a population, the subject is recommended to receive and/or be administered a treatment for prostate cancer. In some embodiments, if the zinc score of the subject is determined to be or be equivalent to a zinc-creatine score of less than 4, the subject is recommended to receive and/or be administered a treatment for prostate cancer. In some embodiments, if the zinc score of the subject is determined to be greater than 85% of the average zinc score of a population, the subject is recommended to receive and/or be administered a treatment for prostate cancer. In some embodiments, if the zinc score of the subject is determined to be or be equivalent to a zinc-creatine score of greater than 7, the subject is recommended not to receive and/or be administered a treatment for prostate cancer. Non-limiting examples of treatments for prostate cancer include radial prostatectomy; chemotherapy; temozolomide; hormone therapy; radiation therapy; external beam radiation therapy; brachytherapy; cryosurgery; high-intensity focused ultrasound; and surgery.

In some embodiments, the treatment for prostate cancer is continued until EPSU samples obtained from the subject are determined to have a zinc score of more than 85% of the average zinc score of a population of healthy adult males. In some embodiments, the treatment for prostate cancer is continued until EPSU samples obtained from the subject are determined to have a zinc score of or equivalent to a zinc-creatinine score of greater than 4.

In some embodiments, the treatment for prostate cancer is continued until EPSU samples obtained from the subject are determined to have a zinc score of more than 85% of the average zinc score of a population of healthy adult males. In some embodiments, the treatment for prostate cancer is continued until EPSU samples obtained from the subject are determined to have a zinc score of or equivalent to a zinc-creatinine score of greater than 7.

In some embodiments, if the zinc score of the subject is determined to be less than 55% but more than 85% of the average zinc score of a population, the subject is recommended undergo further testing and/or monitoring for prostate cancer. In some embodiments, if the zinc score of the subject is determined to be or be equivalent to a zinc-creatine score of less than or equal to 7 but greater than 4, the subject is recommended to undergo further testing and/or monitoring for prostate cancer. Non-limiting examples of testing and/or monitoring for prostate cancer can include, determining the zinc score for a sample obtained from a subject at a later date and determining if the subject's zinc score is changing over time, a PSA test, a biopsy, digital rectal examinations, and monitoring and/or recording of symptoms and the severity of symptoms of prostate cancer. Testing and monitoring for prostate cancer can include testing and/or monitoring at regular intervals. A regular interval can be at least daily, e.g. weekly, monthly, every 2 months, every 3 months, every 6 months, or at other intervals.

The methods and assays described herein may also be used for clinical and self-test assays and instrumentation for diagnosis of prostate cancer. By way of non-limiting example, the methods can be used in conjunction with an assay in which EPSU samples are collected at a clinic. A small volume of the sample can be deposited in a well (e.g. of a 96-well plate) and titrated with a zinc sensor compound in parallel with a non-biological buffer. Fluorescence measurements at defined wavelengths can be taken at each step of the titration using a benchtop fluorimeter. The process can be continued until a peak ratio of sample over buffer fluorescence has been passed. Based on the peak ratio, a measurement of sample zinc concentration can be determined. A separate aliquot of the EPSU sample can be used to determine the concentration of a control substance and the zinc score can be determined. The process can be performed in a clinical scenario, for instance by a laboratory technician, likely involving a comparison to zinc standards, as well as positive and negative controls to ensure the robustness of the measurement. The zinc score can then be compared to the zinc-creatine scores or the average zinc score of a population of healthy adult males as described herein to determine if the subject is at risk of having prostate cancer.

In view of the above descriptions, one skilled in the art can use the methods of the invention in assays and self-test kits. Various alternative embodiments relating to the assays, self-test kits, and other related devices can also be produced, as appreciated by those of skill in the art. Suitable devices are described in U.S. Provisional Application No. 61/358,530, filed Jun. 25, 2010, entitled “Colorimetric Method and Device for Detecting Analyte Quantities in Fluids and Materials,” herein incorporated by reference in its entirety.

In some aspects, the invention described herein is directed to systems (and computer readable media for causing computer systems) for obtaining data from at least one EPSU sample obtained from at least one subject, the system comprising 1) a determination module configured to receive the at least one EPSU sample and perform at least one analysis on the at least one EPSU sample to determine the level of zinc and/or a control substance in the sample; 2) a storage device configured to store data output from the determination module; and 3) a display module for displaying a content based in part on the data output from the determination module, wherein the content comprises a signal indicative of the level of zinc, the level of a control substance, a zinc score and/or an equivalent zinc score.

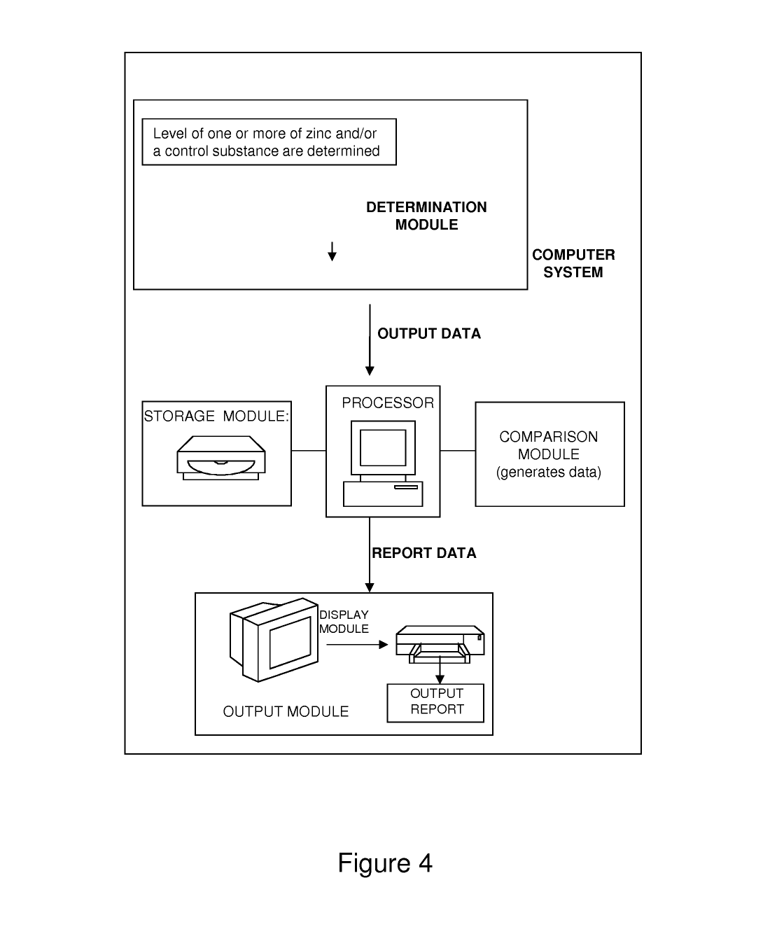

In one embodiment, provided herein is a system comprising: (a) at least one memory containing at least one computer program adapted to control the operation of the computer system to implement a method that includes (i) a determination module configured to receive the at least one EPSU sample and perform at least one analysis on the at least one EPSU sample to determine the level of zinc and/or a control substance in the sample; (ii) a storage module configured to store output data from the determination module; (iii) a computing module adapted to identify from the output data whether the level of zinc and/or a control substance in a EPSU sample obtained from a subject indicates that the zinc score is lower than 85% of the average zinc score in a population of healthy adult male subjects and (iv) a display module for displaying a content based in part on the data output from the determination module, wherein the content comprises a signal indicative of the zinc score and (b) at least one processor for executing the computer program (see FIG. 4).

In one embodiment, provided herein is a system comprising: (a) at least one memory containing at least one computer program adapted to control the operation of the computer system to implement a method that includes (i) a determination module configured to receive the at least one EPSU sample and perform at least one analysis on the at least one EPSU sample to determine the level of zinc and/or a control substance (e.g. creatinine) in the sample; (ii) a storage module configured to store output data from the determination module; (iii) a computing module adapted to identify from the output data whether the level of zinc and/or a control substance in a EPSU sample obtained from a subject indicates that the zinc score is or is equivalent to a zinc-creatinine score of less than 7 and (iv) a display module for displaying a content based in part on the data output from the determination module, wherein the content comprises a signal indicative of the zinc score and (b) at least one processor for executing the computer program (see FIG. 4).

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip.

The term a “computer system” may refer to a system having a computer, where the computer comprises a computer-readable medium embodying software to operate the computer.

The term “software” is used interchangeably herein with “program” and refers to prescribed rules to operate a computer. Examples of software include: software; code segments; instructions; computer programs; and programmed logic.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The functional modules of certain embodiments of the invention include at minimum a determination module, a storage module, a computing module, and a display module. The functional modules can be executed on one, or multiple, computers, or by using one, or multiple, computer networks. The determination module has computer executable instructions to provide e.g., levels of expression products etc. in computer readable form.

The determination module can comprise any system for detecting a signal elicited from an assay to determine the level of zinc and/or a control substance (e.g. creatinine) as described above herein. In some embodiments, such systems can include an instrument, e.g., a SpectraMax M2 fluorescence spectrophotometer (Molecular Devices) described herein for quantitative ZPP1 fluorescence assay measurement of zinc levels. In another embodiment, the determination module can comprise multiple units for different functions, such as quantitative ZPP1-based fluorescence measurement of zinc (e.g. SpectraMax M2) and quantitative measurement of a control substance, e.g. creatinine (e.g. colorimetric assay as detected by the SpectraMax M2).

In some embodiments, the determination system or a further module can be configured to process EPSU samples, e.g. to centrifuge the sample to form a cell-free supernatant for use in the assays described herein.

The information determined in the determination system can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, biomolecule assayed and the level of said biomolecule. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. One of ordinary skill in the art can readily adopt any of the presently known methods for recording information on known media to generate manufactures comprising expression level information.

In some embodiments of any of the systems described herein, the storage module stores the output data from the determination module. In additional embodiments, the storage module stores reference information such as zinc scores in healthy adult male subjects and/or a population of healthy adult male subjects.

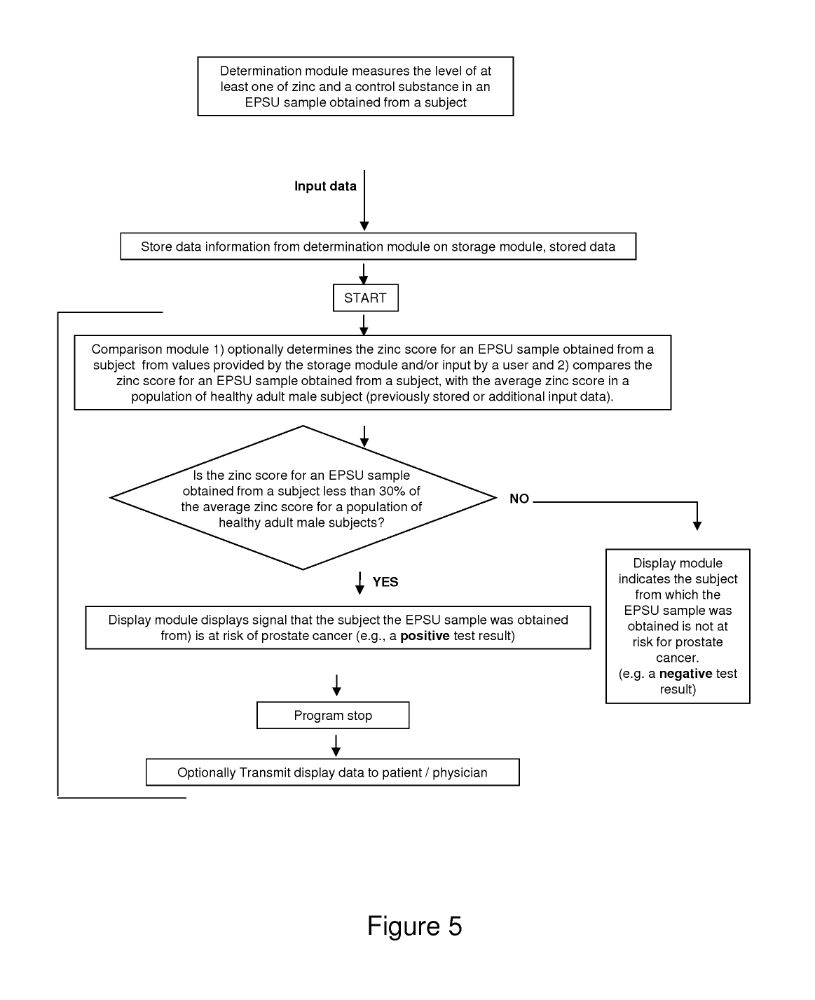

The “computing module” can use a variety of available software programs and formats for computing the zinc score. Such algorithms are well established in the art. A skilled artisan is readily able to determine the appropriate algorithms based on the size and quality of the sample and type of data. The data analysis tools and equations described herein can be implemented in the computing module of the invention. In one embodiment, the computing module further comprises a comparison module, which compares the zinc score in an EPSU sample obtained from a subject as described herein with the average zinc score in a population of healthy adult male subjects (FIG. 5). By way of an example, when the zinc score for an EPSU sample obtained from a subject is determined, a comparison module can compare or match the output data with the average zinc score in a population of healthy adult male subjects. In certain embodiments, the average zinc score in a population of healthy adult male subjects can be pre-stored in the storage module. In some embodiments, during the comparison or matching process, the comparison module can determine whether the zinc score for an EPUS sample obtained from a subject is less than 85% of the average zinc score in a population of healthy adult male subjects. In some embodiments, during the comparison or matching process, the comparison module can determine whether the zinc score for an EPUS sample obtained from a subject is less than 55% of the average zinc score in a population of healthy adult male subjects. In certain embodiments, the average zinc score in a population of healthy adult male subjects can be pre-stored in the storage module. In some embodiments, the comparison module can determine whether the zinc-creatinine score for an EPSU sample obtained from a subject is or is equivalent to a zinc-creatinine score of less than 7. In some embodiments, the comparison module can determine whether the zinc-creatinine score for an EPSU sample obtained from a subject is or is equivalent to a zinc-creatinine score of less than 4. In various embodiments, the comparison module can be configured using existing commercially-available or freely-available software for comparison purpose, and may be optimized for particular data comparisons that are conducted.

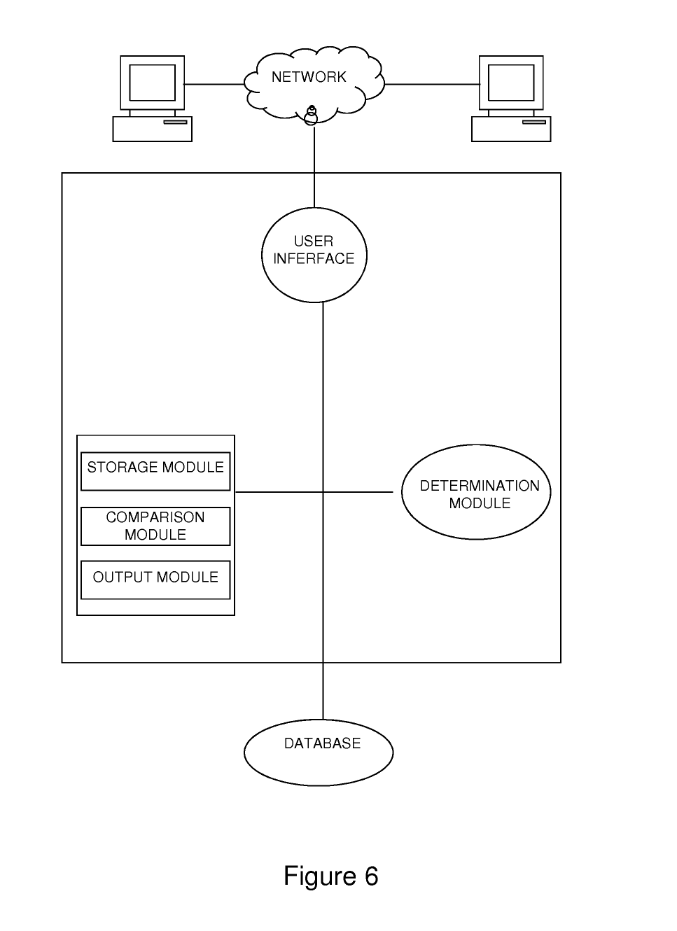

The computing and/or comparison module, or any other module of the invention, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). In some embodiments users can directly access data (via Hypertext links for example) residing on Internet databases using a HTML interface provided by Web browsers and Web servers (FIG. 6).

The computing and/or comparison module provides a computer readable comparison result that can be processed in computer readable form by predefined criteria, or criteria defined by a user, to provide content based in part on the comparison result that may be stored and output as requested by a user using an output module, e.g., a display module.

In some embodiments, the content displayed on the display module can be the level of zinc in the EPSU sample obtained from a subject. In some embodiments, the content displayed on the display module can be the zinc score determined for an EPSU sample obtained from a subject. In some embodiments, the content displayed on the display module can be the zinc-creatinine score determined for the EPSU sample obtained from a subject. In some embodiments, the content displayed on the display module can be an zinc score of second type calculated from the zinc score of a first type determined from EPSU sample obtained from a subject. In some embodiments, the content displayed on the display module can be the zinc score determined from the EPSU sample obtained from a subject as compared to the average zinc score in a population of healthy subjects. In some embodiments, the content displayed on the display module can indicate whether the zinc score determined from the EPSU sample obtained from a subject is less or more than 85% of the average zinc score in a population of healthy adult male subjects. In some embodiments, the content displayed on the display module can indicate whether the zinc score determined from the EPSU sample obtained from a subject is less or more than 55% of the average zinc score in a population of healthy adult male subjects. In some embodiments, the content displayed on the display module can indicate whether the zinc score determined from the EPSU sample obtained from a subject is or is equivalent to a zinc-creatine score of less than 7. In some embodiments, the content displayed on the display module can indicate whether the zinc score determined from the EPSU sample obtained from a subject is or is equivalent to a zinc-creatine score of less than 4. In some embodiments, the content displayed on the display module can indicate whether the subject is at risk of having or developing prostate cancer. In some embodiments, the content displayed on the display module can indicate whether the subject is in need of treatment for prostate cancer. In some embodiments, the content displayed on the display module can indicate whether the subject is recommended to receive further testing and/or monitoring for prostate cancer. In some embodiments, the content displayed on the display module can be a numerical value indicating one of these risks or probabilities. In such embodiments, the probability can be expressed in percentages or a fraction. For example, higher percentage or a fraction closer to 1 indicates a higher likelihood of a subject having prostate cancer. In some embodiments, the content displayed on the display module can be single word or phrases to qualitatively indicate a risk or probability. For example, a word “unlikely” can be used to indicate a lower risk for having or developing prostate cancer, while “likely” can be used to indicate a high risk for having or developing prostate cancer.

In one embodiment of the invention, the content based on the computing and/or comparison result is displayed on a computer monitor. In one embodiment of the invention, the content based on the computing and/or comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the computing/comparison result. It should be understood that other modules of the invention can be adapted to have a web browser interface. Through the Web browser, a user can construct requests for retrieving data from the computing/comparison module. Thus, the user will typically point and click to user interface elements such as buttons, pull down menus, scroll bars and the like conventionally employed in graphical user interfaces.

In some embodiments, the system further comprises a means of inputting a value for the level of one or more of zinc or a control substance determined to be in an EPSU sample obtained from a subject. By way of non-limiting example, the level of zinc can be determined by the determination module of the system while the level of creatinine is determined by a colorimetric assay performed separately from the system described herein ((my. Cat. No. 500701 from Cayman (Ann Arbor, Mich.) or Cat No. ab65340 from AbCam (Cambridge, Mass.). When the level of creatinine is determined, the value for this level can be entered into the computing module of the system and used to determine the zinc score for the EPSU sample obtained from the subject. In some embodiments, the inputting means comprises a keyboard or touchscreen which allows a user to type a value which is accepted by the computing module.

Systems and computer readable media described herein are merely illustrative embodiments of the invention for determining the zinc score for an EPSU sample obtained from a subject, and therefore are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

This invention is further illustrated by the following examples which should not be construed as limiting.

Some embodiments of the technology described herein can be defined according to any of the following numbered paragraphs:
1. A method of treatment comprising:
   determining the level of zinc and a control substance in an expressed prostate secretion urine sample obtained from the subject;
   determining a zinc score, wherein the zinc score is equal to the concentration of zinc multiplied by the control substance concentration;
   administering a treatment for prostate cancer if the score is 85% or less of the average zinc score for a population of healthy adult males and not administering a treatment for prostate cancer if the score is greater than 85% of the average zinc score for a population of healthy adult males.
2. The method of paragraph 1, wherein the level of zinc is determined using ZPP1.
3. The method of any of paragraphs 1-2, wherein the level of zinc is determined using a titration assay.
4. The method of any of paragraphs 1-3, wherein the control substance comprises creatinine.
5. The method of any of paragraphs 1-4, wherein the zinc score is determined by multiplying the level of zinc in uM by the level of creatinine in ug/dL.
6. The method of any of paragraphs 1-5, wherein the treatment for prostate cancer is selected from the group consisting of:
   radial prostatectomy; chemotherapy; temozolomide; hormone therapy; radiation therapy; external beam radiation therapy; brachytherapy; cryosurgery; high-intensity focused ultrasound; and surgery.
7. The method of any of paragraphs 1-6, wherein, if the score is less than 85% and more than 55% of the average zinc score for a population of healthy adult males, the subject is administered further testing or monitoring for prostate cancer.
8. The method of any of paragraphs 1-7, wherein a score of less than 85% of the average zinc score for a population of healthy adult males is equivalent to a zinc-creatinine score of 7 or less.
9. The method of any of paragraphs 1-8, wherein a score of less than 55% of the average zinc score for a population of healthy adult males is equivalent to a zinc-creatine score of 4 or less.
10. An assay comprising:
   contacting a series of solutions of detectable ZPP1 with portions of an expressed prostate secretion urine sample wherein the concentration of ZPP1 increases in a stepwise manner in the series of solutions;
   detecting a signal from ZPP1 in each of the solutions;
   determining, from the signal detected, the level of zinc in the sample;
   determining the level of creatine in the sample;
   determining a zinc score, wherein the zinc score is equal to the level of zinc in uM multiplied by the level of creatine in ug/dL.
11. The assay of paragraph 10, wherein the subject is determined to be at risk of prostate cancer if the score is 85% or less of the average zinc score for a population of healthy adult males.
12. The assay of any of paragraphs 10-11, wherein the subject is at risk of having a high-risk prostate cancer if the zinc score is less than 55% of the average zinc score for a population of healthy adult males.
13. The assay of any of paragraphs 10-12, wherein the zinc score of the subject is determined for a sample obtained at a first date and further comprising determining the zinc score in at least one additional sample obtained at a later date;
   wherein a decrease in the zinc score over time indicates the subject is at risk of having or developing prostate cancer.
14. The assay of any of paragraphs 10-13, wherein a treatment for prostate cancer is administered to a subject determined to be at risk of prostate cancer.
15. The assay of paragraph 14, wherein the treatment for prostate cancer is selected from the group consisting of:
   radial prostatectomy; chemotherapy; temozolomide; hormone therapy; radiation therapy; external beam radiation therapy; brachytherapy; cryosurgery; high-intensity focused ultrasound; and surgery.
16. The assay of any of paragraphs 10-15, wherein, if the score is greater than 85% of the average zinc score for a population of healthy adult males, the subject is recommended to not receive treatment for prostate cancer.
17. The assay of any of paragraphs 10-16, wherein, if the score is less than 85% and more than 55% of the average zinc score for a population of healthy adult males, the subject is recommended to undergo further testing or monitoring for prostate cancer.
18. The assay of any of paragraphs 10-17, further comprising the step of administering a further test or monitoring the subject for prostate cancer if the score is less than 85% and more than 55% of the average zinc score for a population of healthy adult males.
19. The assay of any of paragraphs 10-18, wherein a score of less than 85% of the average zinc score for a population of healthy adult males is equivalent to a zinc-creatinine score of 7 or less.
20. The assay of any of paragraphs 10-19, wherein a score of less than 55% of the average zinc score for a population of healthy adult males is equivalent to a zinc-creatine score of 4 or less.
21. An assay for determining the likelihood of a subject having prostate cancer, the assay comprising:
   determining the level of zinc and a control substance in an expressed prostate secretion urine sample obtained from the subject;
   determining a zinc score, wherein the zinc score is equal to the concentration of zinc multiplied by the control substance concentration;
   wherein the subject is at risk of prostate cancer if the score is 85% or less of the average zinc score for a population of healthy adult males.
22. The assay of paragraph 21, wherein the level of zinc is determined using ZPP1.
23. The assay of any of paragraphs 21-22, wherein the level of zinc is determined using a titration assay.
24. The assay of any of paragraphs 21-23, wherein the control substance comprises creatinine.
25. The assay of any of paragraphs 21-24, wherein the zinc score is determined by multiplying the level of zinc in uM by the level of creatinine in ug/dL.
26. The assay of any of paragraphs 21-25, wherein the subject is at risk of having a high-risk prostate cancer if the zinc score is less than 55% of the average zinc score for a population of healthy adult males.
27. The assay of any of paragraphs 21-26, wherein the zinc score of the subject is determined for a sample obtained at a first date and further comprising determining the zinc score in at least one additional sample obtained at a later date;
   wherein a decrease in the zinc score over time indicates the subject is at risk of having or developing prostate cancer.
28. The assay of any of paragraphs 21-27, wherein if the zinc score is less than 55% of the average zinc score for a population of healthy adult males, the subject is recommended to be treated with a treatment selected from the group consisting of:
   radial prostatectomy; chemotherapy; temozolomide; hormone therapy; radiation therapy; external beam radiation therapy; brachytherapy; cryosurgery; high-intensity focused ultrasound; and surgery.
29. The assay of any of paragraphs 21-28, wherein, if the score is greater than 85% of the average zinc score for a population of healthy adult males, the subject is recommended to not receive treatment for prostate cancer.
30. The assay of any of paragraphs 21-29, wherein, if the score is less than 85% and more than 55% of the average zinc score for a population of healthy adult males, the subject is recommended to undergo further testing or monitoring for prostate cancer.
31. An assay comprising:
   determining the level of zinc and a control substance in an expressed prostate secretion urine sample obtained from the subject;
   determining a zinc score, wherein the zinc score is equal to the concentration of zinc multiplied by the control substance concentration;
   wherein the subject has an increased likelihood of having prostate cancer if the score is or is equivalent to a zinc-creatinine score of 7 or less.
32. The assay of paragraph 31, wherein the level of zinc is determined using ZPP1.
33. The assay of any of paragraphs 31-32, wherein the level of zinc is determined using a titration assay.
34. The assay of any of paragraphs 31-33, wherein the control substance comprises creatinine.
35. The assay of any of paragraphs 31-34, wherein the zinc score is determined by multiplying the level of zinc in uM by the level of creatinine in ug/dL.
36. The assay of any of paragraphs 31-35, wherein the subject is at risk of having a high-risk prostate cancer if the zinc score is or is equivalent to a zinc-creatinine score of less than 4.
37. The assay of any of paragraphs 31-36, wherein the zinc score of the subject is determined for a sample obtained at a first date and further comprising determining the zinc score in at least one additional sample obtained at a later date;
   wherein a decrease in the zinc score over time indicates the subject is at risk of having or developing prostate cancer.
38. The assay of any of paragraphs 31-37, wherein, if the zinc score is or is equivalent to a zinc-creatinine score of less than 4, the subject is recommended to be treated with a treatment selected from the group consisting of:
   radial prostatectomy; chemotherapy; temozolomide; hormone therapy; radiation therapy; external beam radiation therapy; brachytherapy; cryosurgery; high-intensity focused ultrasound; and surgery.
39. The assay of any of paragraphs 31-38, wherein, if the score is or is equivalent to a zinc-creatinine score of 7 or more, the subject is recommended to not receive treatment for prostate cancer.
40. The assay of any of paragraphs 31-39, wherein, if the zinc score is or is equivalent to a zinc-creatinine score of more than 4 and less than 7, the subject is recommended to undergo further testing or monitoring for prostate cancer.
41. A system for obtaining data from at least one expressed prostate secretion urine sample obtained from at least one subject, the system comprising:
   a determination module configured to receive the at least one expressed prostate secretion urine sample and perform at least one analysis on the at least one expressed prostate secretion urine sample to determine a zinc score;
   a storage device configured to store data output from said determination module; and
   a display module for displaying a content based in part on the data output from said determination module, wherein the content comprises a signal indicative of the zinc score.
42. The system of paragraph 41, wherein the system further comprises a means of inputting a value for the level of one or more of zinc or a control substance determined to be in the expressed prostate secretion urine sample.
43. The system of any of paragraphs 41-42, wherein the content displayed on said display module further comprises a signal indicative of the subject being at risk of having or developing prostate cancer if the zinc score is determined to be less than 85% of the average zinc score in a population of healthy adult males.
44. The system of any of paragraphs 41-43, wherein the content displayed on said display module further comprises a signal indicative of the subject being at risk of having or developing prostate cancer if the zinc score is determined to be or be equivalent to a zinc-creatinine score of less than 7.
45. The system of any of paragraphs 41-44, wherein the content displayed on said display module further comprises a signal indicative of the subject being recommended to further testing or monitoring for prostate cancer.
46. The system of any of paragraphs 41-45, wherein the content displayed on said display module further comprises a signal indicative of the subject being at risk of having or developing prostate cancer if the zinc score is determined to be less than 85% of the average zinc score in a population of healthy adult males.
47. The system of any of paragraphs 41-46, wherein the content displayed on said display module further comprises a signal indicative of the subject being at risk of having or developing prostate cancer if the zinc score is determined to be or be equivalent to a zinc-creatinine score of less than 4.
48. The system of any of paragraphs 41-47, wherein the content displayed on said display module further comprises a signal indicative of the subject being recommended to receive treatment for prostate cancer.
49. A method for determining the likelihood of a subject having prostate cancer, the method comprising:
   determining the level of zinc and a control substance in an expressed prostate secretion urine sample obtained from the subject;
   determining a zinc score, wherein the zinc score is equal to the concentration of zinc multiplied by the control substance concentration;
   wherein the subject is at risk of prostate cancer if the score is 85% or less of the average zinc score for a population of healthy adult males.
50. The method of paragraph 49, wherein the level of zinc is determined using ZPP1.
51. The method of any of paragraphs 49-50, wherein the level of zinc is determined using a titration assay.
52. The method of any of paragraphs 49-51, wherein the control substance comprises creatinine.
53. The method of any of paragraphs 49-52, wherein the zinc score is determined by multiplying the level of zinc in uM by the level of creatinine in ug/dL.
54. The method of any of paragraphs 49-53, wherein the subject is at risk of having a high-risk prostate cancer if the zinc score is less than 55% of the average zinc score for a population of healthy adult males.
55. The method of any of paragraphs 49-54, wherein the zinc score of the subject is determined for a sample obtained at a first date and further comprising determining the zinc score in at least one additional sample obtained at a later date;
   wherein a decrease in the zinc score over time indicates the subject is at risk of having or developing prostate cancer.
56. The method of any of paragraphs 49-55, wherein if the zinc score is less than 55% of the average zinc score for a population of healthy adult males, the subject is recommended to be treated with a treatment selected from the group consisting of:
   radial prostatectomy; chemotherapy; temozolomide; hormone therapy; radiation therapy; external beam radiation therapy; brachytherapy; cryosurgery; high-intensity focused ultrasound; and surgery.
57. The method of any of paragraphs 49-56, wherein, if the score is greater than 85% of the average zinc score for a population of healthy adult males, the subject is recommended to not receive treatment for prostate cancer.
58. The method of any of paragraphs 49-57, wherein, if the score is less than 85% and more than 55% of the average zinc score for a population of healthy adult males, the subject is recommended to undergo further testing or monitoring for prostate cancer.
59. A method comprising:
   determining the level of zinc and a control substance in an expressed prostate secretion urine sample obtained from the subject;
   determining a zinc score, wherein the zinc score is equal to the concentration of zinc multiplied by the control substance concentration;
   wherein the subject has an increased likelihood of having prostate cancer if the score is or is equivalent to a zinc-creatinine score of 7 or less.
60. The method of paragraph 59, wherein the level of zinc is determined using ZPP1.
61. The method of any of paragraphs 59-60, wherein the level of zinc is determined using a titration assay.
62. The method of any of paragraphs 59-61, wherein the control substance comprises creatinine.
63. The method of any of paragraphs 59-62, wherein the zinc score is determined by multiplying the level of zinc in uM by the level of creatinine in ug/dL.
64. The method of any of paragraphs 59-63, wherein the subject is at risk of having a high-risk prostate cancer if the zinc score is or is equivalent to a zinc-creatinine score of less than 4.
65. The method of any of paragraphs 59-64, wherein the zinc score of the subject is determined for a sample obtained at a first date and further comprising determining the zinc score in at least one additional sample obtained at a later date;
   wherein a decrease in the zinc score over time indicates the subject is at risk of having or developing prostate cancer.
66. The method of any of paragraphs 59-65, wherein, if the zinc score is or is equivalent to a zinc-creatinine score of less than 4, the subject is recommended to be treated with a treatment selected from the group consisting of:
   radial prostatectomy; chemotherapy; temozolomide; hormone therapy; radiation therapy; external beam radiation therapy; brachytherapy; cryosurgery; high-intensity focused ultrasound; and surgery.
67. The method of any of paragraphs 59-66, wherein, if the score is or is equivalent to a zinc-creatinine score of 7 or more, the subject is recommended to not receive treatment for prostate cancer.
68. The method of any of paragraphs 59-67, wherein, if the zinc score is or is equivalent to a zinc-creatinine score of more than 4 and less than 7, the subject is recommended to undergo further testing or monitoring for prostate cancer.

### EXAMPLES

The early detection of prostate cancer is a life-saving event in patients harboring potentially aggressive disease. With the development of malignancy there is a dramatic reduction in the zinc content of prostate tissue associated with the inability of cancer cells to accumulate the ion.

In the current study, endogenous zinc was utilized as a diagnostic biomarker for prostate cancer. A novel fluorescent sensor for mobile zinc (ZPP1) was employed to measure the concentration of zinc in thirty-nine patient samples of expressed prostatic secretion (EPS) in urine. The probability of classifying a subject as benign, low-risk, or high-risk as functions of the diagnostic test results was estimated using a non-informative prior Bayesian approach. Permutation tests and other non-parametric tests were also used.

A significant trend in zinc score with disease and with disease risk (P=0.03), and lack of a significant correlation between zinc score and PSA. Demonstrated herein is that the proposed diagnostic is potentially superior to PSA for detecting high-risk disease.

In the examples provided herein, zinc was utilized as a prostate cancer biomarker. Described herein is an assay that allows quantification of biological mobile zinc. The assay uses a zinc sensor (ZPP1) with a unique turn-on, biphasic response to zinc (15, 16). This property of ZPP1 opens up the opportunity to accurately quantify zinc in prostate cancer samples, using simple titration of the sensor (15, 16). More importantly, the assay described herein showed the possibility for risk assessment in cancer patients. The rapid development, validation, and introduction of this technology into the clinic has the potential to significantly increase the sensitivity and specificity of prostate cancer detection, as well as to provide an additional tool for disease staging.

### Example 1

### Materials And Methods

Chemical Reagents.

The cell membrane-permeable fluorescent Zn²⁺ sensor ZPP1 was prepared and characterized as previously described (15).

Patients.

Patient characteristics are reported in Table 1. All samples were procured using protocols approved by the EVMS Institutional Review Board, and all applicable NIH guidelines and HIPAA regulations were followed. No personal information or identifiers were available to the laboratory investigators other than diagnosis, age, and lab results. All experiments were performed with approval by the institutional review board of the Massachusetts General Hospital and in accordance with an assurance filed with and approved by the U.S. Department of Health and Human Services.

Clinical Samples.

The expressed prostate secretion fluids were obtained following gentle prostate massage during digital rectal examination prior to biopsy. The massage consists of three strokes on each side of the median sulcus of the prostate. This procedure forces the expressed fluid from the glandular network of the prostate directly into the urethra. Urine (10-20 ml) containing the EPS was then collected from each individual and stored on ice. At the bio-repository, 9 ml of each sample were centrifuged to remove the cell pellet/particulate, and tubes of 0.5 ml, 1 ml (X4), and 4.5 ml were stored at −80° C. Cell pellets/sediment was also stored.

It should be emphasized that all of the EPS urine samples were obtained from men at the clinic just prior to their biopsy procedure. Hence, even those individuals with no evidence of cancer, and those with biopsy confirmed BPH, generally will have serum PSA values in the 2.5-10 ng/ml ranges. The EPS samples are initially classified using the currently accepted risk stratification system and include the results of a biopsy with a minimum of 12 cores. These classifications are presented in Table 2.

### Measurement of EPS-Urine Zinc Content (“ZPP1 Test”)

Measurement of Zinc Concentration Using ZPP1 Titration.

Titrations were performed as previously described (15, 17). Briefly, EPS urine samples were centrifuged at 2500 rpm for 5 min and diluted (1:4) in HEPES/KCl buffer (25 mmol/L HEPES and 100 mmol/L KCl; pH 7.0). For fluorescence analysis of zinc, 0.1 ml diluted EPS urine samples were added to 96-well plates. ZPP1 was titrated into the sample to achieve stepwise increments in ZPP1 concentration. At each step, the fluorescence was measured (excitation, 505 nm; emission, 532 nm) using a SpectraMax M2 fluorescence spectrophotometer (Molecular Devices). For each measurement, the fluorescence of buffer containing and equivalent amount of ZPP1 alone was subtracted from that of the sample. Because of the chemical properties of ZPP1, the titration curve gives a maximum when its concentration is exactly half that of the mobile zinc in the test solution. The zinc concentration can then be calculated according to the formula:

[Zn²⁺]=2[ZPP1]ₘₐₓ

In accord with the literature (15), initial validation experiments in buffer confirmed that the ZPP1 concentration at the peak fluorescence equals half of the zinc concentration in the sample and that the measurements were linear over a range of 0.02-5 mM [Zn²⁺].

Determination of Zinc Score.

The zinc scores were obtained by technicians blinded to the subjects' disease category. To determine zinc score, the EPS-urine zinc content (μM) of each patient was multiplied by creatinine concentration (μg/dL), in order to account for differences in the prostatic fluid fraction of the samples.

To measure the creatinine level in EPS urine, a commercially available kit from Cayman (Ann Arbor, Mich.) was used.

The zinc score was not obtained for one subject (PSA=65, high-risk), for reasons independent of the subject's disease category. This subject was omitted from the analysis below.

Statistical Analysis.

Data analysis was performed using the R package for statistical analysis and graphics (18). The diagnostic biomarker (PSA or zinc score) values were assumed to be normally distributed by disease category (Benign, Low-Risk, High-Risk), with means and standard deviations that depended on the category. The proportions of subjects in each category were used as the prior probability of category classification. For the category means and standard deviations, the usual non-informative priors were used, i.e., proportional to a constant and to the reciprocal standard deviation, respectively. Since under these assumptions the posterior distributions of the biomarker value given the category will have the usual t-distributions for simple random samples (19), it is straightforward to compute numerically the posterior probabilities of category classification as functions of the biomarker values.

Strong a-priori reasons existed to expect a monotone ordering of the three group mean zinc scores, with non-cancer highest, high-risk lowest, and low-risk in between. A nonparametric test of this trend was constructed as follows. First, a non-parametric p-value was determined for the maximum absolute difference in the three means from the appropriate permutation distribution. Next, it was noted that, conditional on this maximum absolute difference, the probability that the second largest mean is the low-risk mean is ⅓. So the permutation p-value was divided by three to get the final result.

The significance of the correlations between zinc score and PSA was combined and within-group using the appropriate permutation distributions of the Pearson correlation and compared the performance of PSA and the proposed zinc score as diagnostic tests using McNemar's test (20).

### Example 2

### Results

Sensitivity of the ZPP1 Assay to Zinc Content and Disease State Using Human EPS-Urine.

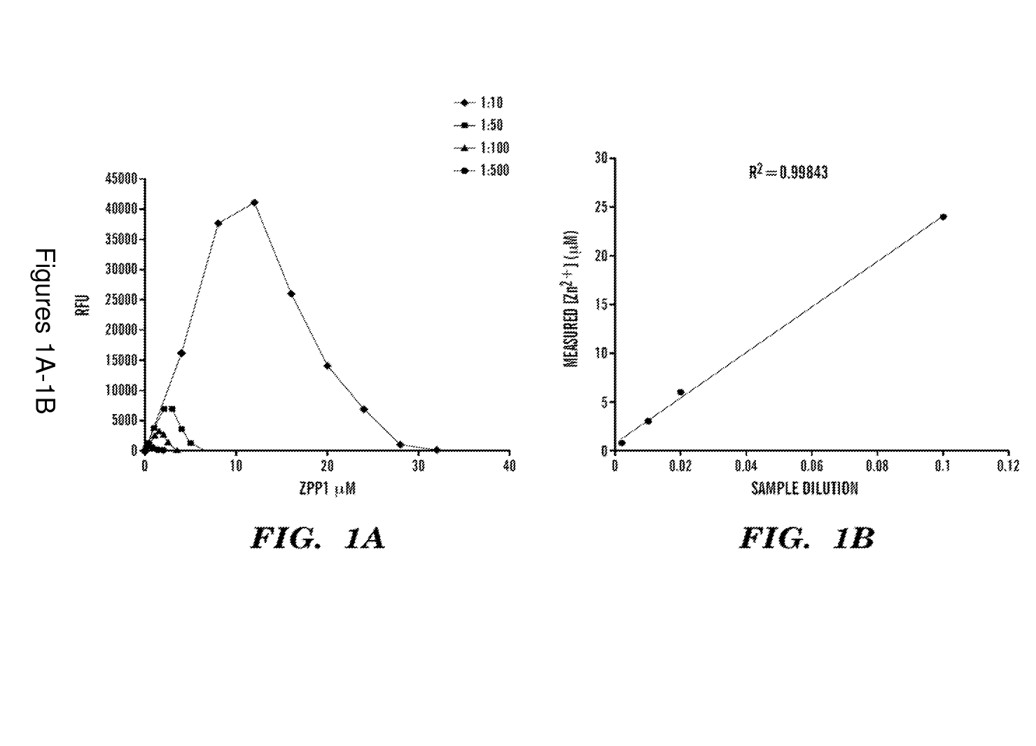

Before assessing the diagnostic value of the ZPP1 test for prostate cancer, the sensitivity of the assay to zinc concentration in EPS-urine was first determined. Serial dilutions of an EPS-urine sample from a non-cancer patient were prepared and their zinc concentrations measured by ZPP1 titration (FIG. 1A). A high linear correlation (R²=0.998) was found between sample dilution and the zinc concentration measured using the ZPP1 assay over the entire range of tested dilutions ([Zn²⁺]=800 nM-25 μM; FIG. 1B). This result suggested that the assay described herein could be used to accurately quantify sub-micromolar zinc concentrations in EPS-urine. Furthermore, since the expected measurements of EPS-urine zinc content in the entire patient population fall within the tested range, the method described herein could be used to probe for differences as a function of disease state (FIG. 1B).

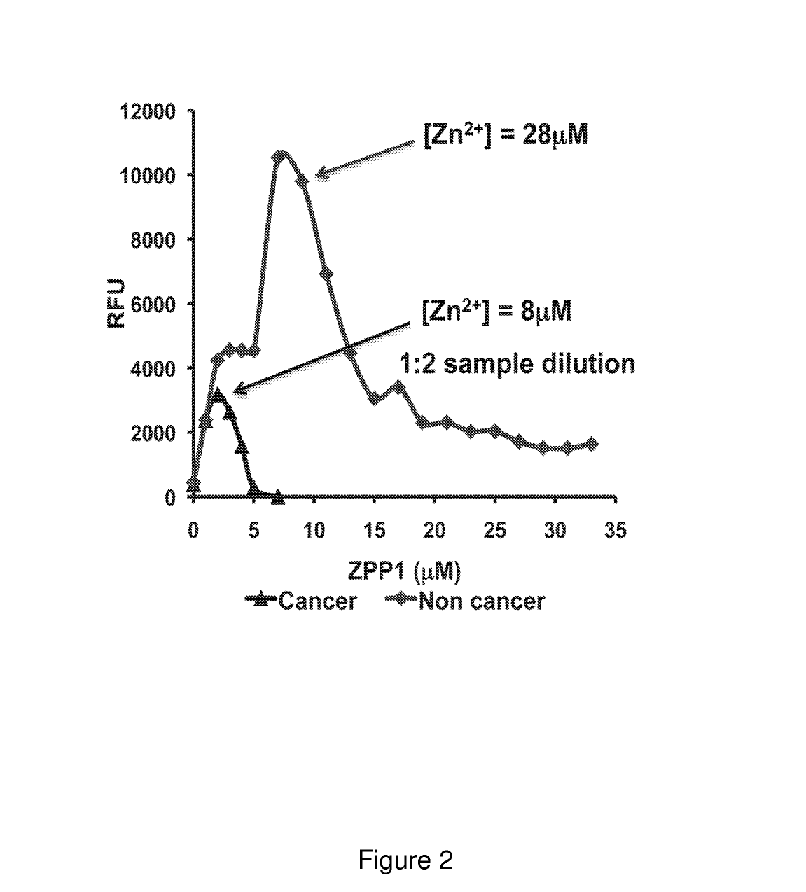

Next, it was determined whether the ZPP1 test could differentiate between non-cancer and cancer based on the zinc content of EPS-urine. EPS-urine samples were pooled from non-cancer and cancer patients (n=25 in each pool). The non-cancer pool included samples from BPH and normal subjects, whereas the cancer pool included samples representative of the entire spectrum of the disease. Using the ZPP1 test, a 3.5-fold reduction was detected in the zinc content of EPS-urine from cancer patients relative to non-cancer controls (FIG. 2).

These results suggested that the ZPP1 test could report on the zinc content of human EPS-urine and, on this basis, has the capacity to differentiate between cancer and non-cancer. Having validated the assay in these studies, risk stratification experiments were performed in a limited number of patient samples.

Application of the ZPP1 Test for Prostate Cancer Diagnosis and Staging.

To investigate the value of the ZPP1 test as a clinical diagnostic and staging tool, EPS urine samples were collected from non-cancer/benign (n=15), low-risk cancer (n=12), and high-risk cancer (n=12) patients, as determined by prostate biopsy. Each sample was assigned a zinc score based on the concentration of zinc obtained from the ZPP1 assay and taking into account the creatinine content of the sample. The mean zinc scores were 7.5, 6.4 and 2.8, for non-cancer, low-risk and high-risk, respectively. A test of the hypothesis that the group means were different revealed no significance (F(2,35)=2.4, P=0.11). However, there was strong a-priori reason to expect the zinc score to decrease monotonically with disease severity. The range of these values is 4.7 (P=0.09, permutation test). However, in addition to a range of this magnitude, the central mean was observed to be low-risk, as predicted. The probability of this is ⅓, so the final p-value for the hypothesized trend in group-mean zinc scores is 0.03. The zinc score was not significantly correlated with PSA, either overall or for any of the three groups considered separately (permutation tests).

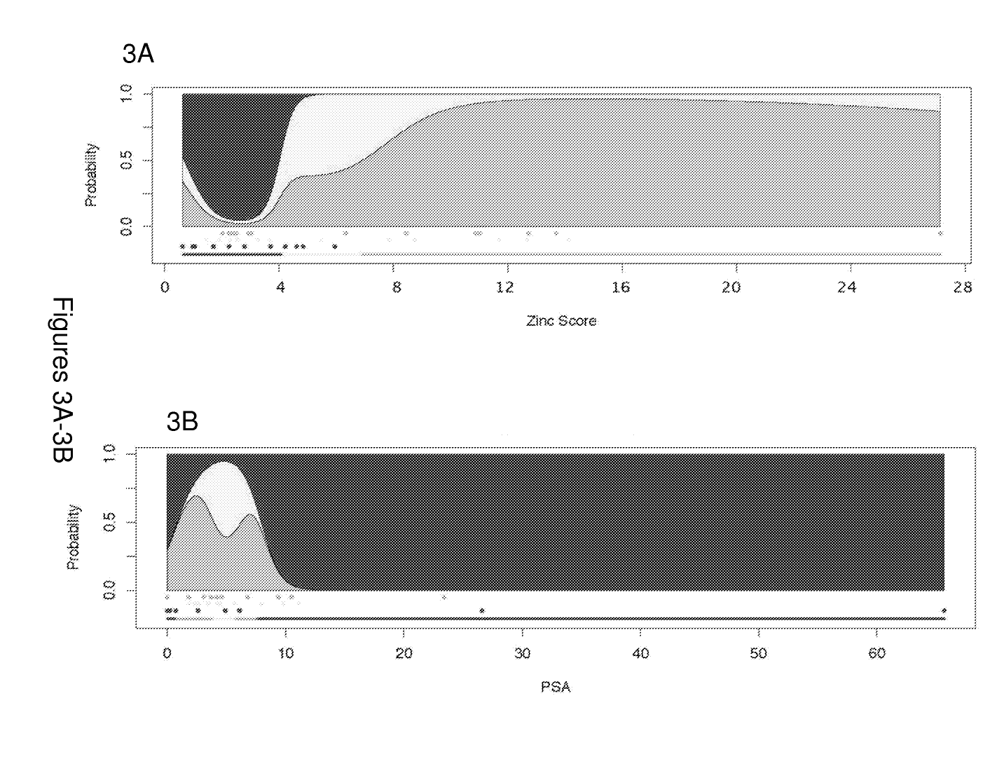

In order to illustrate the potential of zinc score as a predictor of disease category, the following statistical model was considered. Assume that zinc scores are normally distributed given the category, with means and standard deviations that depend on the category. For a Bayesian model with the usual non-informative priors on the normal distribution parameters, it is clear that the posterior distributions of the mean zinc scores given category have t-distributions (19). It is assumed here that the prior probability of a zinc score coming from a subject in a particular disease category is equal to the proportion of subjects in our sample observed to be in this category. With these assumptions, it is straightforward to calculate the posterior probability of category assignment as a function of zinc score, and also as a function of PSA. The results of these calculations are given in FIG. 3. From this Figure, it appears that the usual threshold of 4.0 for PSA seems to be reasonable. For the zinc score, a threshold of 7 to separate malignant from benign, and a second threshold of 4 to separate low-grade from high-grade appear to be reasonable.

The zinc score and PSA were compared for distinguishing a benign condition from malignant disease (Table 3). The error fraction was less for the zinc score (14/38) than for the PSA (20/38), although not significantly so (P=0.30, McNemar's test). However, there is a much greater difference in the diagnostic test performance than these comparable overall error rates suggest. The PSA test makes almost twice as many (13 vs. seven) false-negative predictions as false-positives, whereas the zinc score has only about half as many false-negatives as false-positives (five vs. nine). The false-negative cases from the PSA test divide nearly evenly between high- and low-risk (seven high-risk, six low-risk). From FIG. 3, it is apparent that PSA is not useful for separating high-risk from low-risk malignancies. On the other hand, the five false-negative cases from the zinc score test are all low-risk. It is also notable that eight of the nine false-positives for the zinc score test are classed as high risk and the total number of false-positive readings represented 37.5% of the combined low- and high-risk cancer-diagnosed patients (Table 4). The definition of these samples as false positives, however, can be questioned, taking into account that risk assessment in our study was based on biopsy- and not post-prostatectomy samples. Because, in 30-40% of the cases (21-23), Gleason grade, and therefore risk, are upgraded after prostatectomy, the 40% incidence of “false positives” in the data set described herein could actually represent under-diagnosed true positives.

### Example 3

### Discussion

The ambiguity associated with the current diagnostic tests for prostate cancer forces many men to either undergo unnecessary invasive and emotionally taxing treatment or remain undiagnosed. This dilemma has caused many clinicians and scientists to challenge the notion that testing for prostate cancer is warranted due to the slow-growing nature of the malignancy and its reduced influence on the overall survival of older men with a shorter life expectancy. However, recent trials have affirmed the life-saving value of early diagnosis, especially in younger men (3, 24). Because, according to the American Cancer Society, 1 in 35 men in the U.S. will die of prostate cancer, there should be no debate about the need for an effective and reliable diagnostic tool as a facilitator of successful therapy.

In response to this need, there is described herein a novel zinc-based diagnostic test for prostate cancer, which is strictly quantitative and predictive of disease aggressiveness.

The approach described herein centers around a prostate cancer biomarker which has not fulfilled its promise in a clinical setting, owing to the absence of appropriate clinically relevant tools. This biomarker is most attractive because, whereas the concentration of mobile reactive zinc in the healthy prostate is the highest of all soft tissues in the body, there is a dramatic reduction in prostatic zinc content with the development of malignancy. This effect is specific to prostate cancer and can resolve the differential diagnosis between cancer and benign conditions, such as BPH (25) and prostatitis (26).

The presented diagnostic approach takes advantage of a recently described zinc-sensing fluorescent probe (15) and a simple high-throughput method for measuring zinc concentration in biological samples (15, 17). The method described herein provides information of unprecedented value, since none of the current diagnostic methods can accurately and noninvasively define disease aggressiveness. In spite of a seemingly high false-positive rate of the assay, it is emphasized that 89% of these false positives were classified by as high risk. In addition, as mentioned above, 30-40% of all cases are reclassified based on postprostatectomy histopathology to a higher grade (21-23). We believe that the test described herein will identify these under-diagnosed cases. A further advantage that cannot be underestimated is the ease, rapidity, and convenience with which this test can be performed. In addition, obtaining samples from the patients can be easily incorporated into the routine screening protocol, as a supplement to the DRE procedure. These advantages underscore the likelihood that the test will find clinical application as an addition to the established screening tools. In its totality, this new diagnostic paradigm could adequately address the critical need of risk prediction in the management of prostate cancer.

### Example 4

### References

1. Jemal A, Murray T, Ward E, et al. Cancer statistics, 2005. CA Cancer J Clin 2005; 55: 10-30.
2. Mannuel H D, Hussain A. Evolving role of surgery, radiation, hormone therapy, and chemotherapy in high-risk locally advanced prostate cancer. Clin Genitourin Cancer 2006; 5: 43-9.
3. Parker P M, Rice K R, Sterbis J R, et al. Prostate cancer in men less than the age of 50: a comparison of race and outcomes. Urology; 78: 110-5.
4. Fitzsimons N J, Sun L, Moul J W. Medical technologies for the diagnosis of prostate cancer. Expert Rev Med Devices 2007; 4: 227-39.
5. Smith D S, Humphrey P A, Catalona W J. The early detection of prostate carcinoma with prostate specific antigen: the Washington University experience. Cancer 1997; 80: 1852-6.
6. Thompson I M, Pauler D K, Goodman P J, et al. Prevalence of prostate cancer among men with a prostate-specific antigen level < or =4.0 ng per milliliter. N Engl J Med 2004; 350: 2239-46.
7. Thompson I M, Ankerst D P, Chi C, et al. Assessing prostate cancer risk: results from the Prostate Cancer Prevention Trial. J Natl Cancer Inst 2006; 98: 529-34.
8. Masters J G, Keegan P E, Hildreth A J, Greene D R. Free/total serum prostate-specific antigen ratio: how helpful is it in detecting prostate cancer? Br J Urol 1998; 81: 419-23.
9. Vickers A J, Till C, Tangen C M, Lilja H, Thompson I M. An empirical evaluation of guidelines on prostate-specific antigen velocity in prostate cancer detection. J Natl Cancer Inst; 103: 462-9.
10. Nash A F, Melezinek I. The role of prostate specific antigen measurement in the detection and management of prostate cancer. Endocr Relat Cancer 2000; 7: 37-51.
11. Mawson C A, Fischer M I. The occurrence of zinc in the human prostate gland. Can J Med Sci 1952; 30: 336-9.
12. Costello L C, Franklin R B. Prostatic fluid electrolyte composition for the screening of prostate cancer: a potential solution to a major problem. Prostate Cancer Prostatic Dis 2009; 12: 17-24.
13. Cortesi M, Chechik R, Breskin A, et al. Evaluating the cancer detection and grading potential of prostatic-zinc imaging: a simulation study. Phys Med Biol 2009; 54: 781-96.
14. Cortesi M, Fridman E, Volkov A, et al. Clinical assessment of the cancer diagnostic value of prostatic zinc: a comprehensive needle-biopsy study. Prostate 2008; 68: 994-1006.
15. Mang X A, Hayes D, Smith S J, Friedle S, Lippard S J. New strategy for quantifying biological zinc by a modified zinpyr fluorescence sensor. J Am Chem Soc 2008; 130: 15788-9.
16. Buccella D, Horowitz J A, Lippard S J. Understanding zinc quantification with existing and advanced ditopic fluorescent Zinpyr sensors. J Am Chem Soc 2011; 133: 4101-14.
17. Ghosh S K, Kim P, Zhang X A, et al. A novel imaging approach for early detection of prostate cancer based on endogenous zinc sensing. Cancer Res 2010; 70: 6119-27.
18. Team R D C. A Language and Environment for Statistical Computing. Vienna, Austria: R Foundation for Statistical Computing; 2011.
19. Box G E, Tiao G C. Bayesian inference in statistical analysis. Reading, Mass.: Addison-Wesley Pub. Co.; 1973.
20. Lehmann E L. Nonparametrics: Statistical Methods Based on Ranks: Holden-Day; 1975.
21. Corcoran N M, Hong M K, Casey R G, et al. Upgrade in Gleason score between prostate biopsies and pathology following radical prostatectomy significantly impacts upon the risk of biochemical recurrence. BJU Int.
22. Pinthus J H, Witkos M, Fleshner N E, et al. Prostate cancers scored as Gleason 6 on prostate biopsy are frequently Gleason 7 tumors at radical prostatectomy: implication on outcome. J Urol 2006; 176: 979-84; discussion 84.
23. Thomas C, Pfirrmann K, Pieles F, et al. Predictors for clinically relevant Gleason score upgrade in patients undergoing radical prostatectomy. BJU Int.
24. Varkarakis J, Pinggera G M, Sebe P, Berger A, Bartsch G, Horninger W. Radical retropubic prostatectomy in men younger than 45 years diagnosed during early prostate cancer detection program. Urology 2004; 63: 337-41.
25. Zaichick V, Sviridova T V, Zaichick S V. Zinc in the human prostate gland: normal, hyperplastic and cancerous. Int Urol Nephrol 1997; 29: 565-74.
26. Zaichick V Y, Sviridova T V, Zaichick S V. Zinc concentration in human prostatic fluid: normal, chronic prostatitis, adenoma and cancer. Int Urol Nephrol 1996; 28: 687-94.

TABLE 1
Patient Characteristics

| | | | tumor | | |
|---|---|---|---|---|---|
| | | gleason | volume | | |
| disease state | stage | score | (cm³) | Risk/disease | psa |
| Benign | na | na | 0 | PIN | 0 |
| | na | na | 0 | BPH | 0 |
| | na | na | 0 | BPH | 4.6 |
| | na | na | 0 | BPH | 0 |
| | na | na | 0 | BPH | 0 |
| | na | na | 0 | BPH | 4.6 |
| | na | na | 0 | N | 1.8 |
| | na | na | 0 | N | 0 |
| | na | na | 0 | PIN | 3.7 |
| | na | na | 0 | BPH | 3.1 |
| | na | na | 0 | N | 6.8 |
| | na | na | 0 | N | 4.2 |
| | na | na | 0 | N | 9.4 |
| | na | na | 0 | N | 10.5 |
| | na | na | 0 | PIN | 23.4 |
| Low-risk | T2c | 3 + 3 | 0 | L | 5.7 |
| | CT2ANXMX | 3 + 3 | 0 | L | 4.2 |
| | CT2ANXMX | 3 + 3 | 0 | L | 2.5 |
| | CT1CNXMX | 3 + 3 | 0 | L | 1.8 |
| | CT1CNXMX | <6 | 0 | L | 2.6 |
| | CT1CNXMX | 3 + 3 | 0 | L | 3.5 |
| | CT2ANXMX | 3 + 3 | 0 | L | 7.93 |
| | CT1CNXMX | 3 + 3 | 0 | L | 4.52 |
| | T2c | 3 + 3 | 0 | L | 3.4 |
| | T2c | 3 + 4 | 0 | L | 2.3 |
| | T2c | 3 + 3 | 0 | L | 9.8 |
| | CT1CNXMX | 3 + 3 | 0 | L | 11.1 |
| Intermediate/ | Unknown | 3 + 4 | 0 | I | 8.8 |
| High-risk | T2c | 4 + 3 | 15 | I | 2.6 |
| | T2c | 4 + 3 | 0 | I | 4.9 |
| | T2a | 4 + 4 | 2 | H | 6.1 |
| | T2c | 4 + 4 | 7 | H | 0 |
| | T3a | 3 + 4 | 25 | H | 0 |
| | CT2ANXMX | 4 + 4 | 0 | H | 26.6 |
| | CT1CNXMX | 4 + 5 | 0 | H | 0.2 |
| | CT3BNXMX | 4 + 3 | 0 | H | 0 |
| | CT3BNXMX | 4 + 3 | 0 | H | 0 |
| | CT2BNXMX | 4 + 5 | 0 | H | 0.72 |
| | CT2CNXMX | 5 + 5 | 0 | H | 65.7 |

Abbreviations:
N = no detected disease;
L, I, or H refer to risk stratifications (I = intermediate; L = low; H = high).

TABLE 2
Clinical Definitions

| Risk | Definition |
|---|---|
| Benign | Normal: prostate volume <30 cc, |
| | normal voiding history, |
| | PSA 2.5-10, |
| | negative prostate biopsy, |
| | BPH: prostate volume >30 cc, |
| | bladder outlet obstructive symptoms with AUA |
| | symptom index >14, |
| | biopsy negative; |
| | PSA 2.5-10 |
| Low-Risk | PSA <10, |
| | Gleason 3 + 3 or less, |
| | clinical stage T2a or less, |
| | <30% biopsies core positive |
| Intermediate-Risk | Gleason 4 + 3 or 3 + 4, or |
| | stage T2b or T2c, |
| | or PSA 10-20, |
| | 30-50% biopsies core positive |
| High-Risk | PSA >10, |
| | Gleason score 4 + 4 or higher, |
| | >50% biopsies core positive, |
| | any T stage with the Gleason score noted |

TABLE 3
Predictions vs. actual disease state for zinc score and PSA.
Note that the 5 malignancies misclassified as benign by the
zinc score were all low-risk. Of the 13 malignancies
misclassified as benign by PSA, 7 were high-grade.

| Prediction | |
|---|---|
| Zinc Score | PSA |

| Disease State | Benign | Malignant | Benign | Malignant |
|---|---|---|---|---|
| Benign | 6 | 9 | 8 | 7 |
| Malignant | 5 | 18 | 13 | 10 |

TABLE 4
Results of using zinc score to classify subjects, with thresholds of
≦7 for malignancy, and ≦4 for high-risk malignancy

| | Prediction | | |
|---|---|---|---|
| Disease State | Benign | Low-risk | High-risk |
| Benign | 6 | 1 | 8 |
| Low-risk | 5 | 1 | 6 |
| High-risk | 0 | 4 | 7 |

1. A method of treatment comprising:
determining the level of zinc and a control substance in an expressed prostate secretion urine sample obtained from subject;
determining a zinc score, wherein the zinc score is equal to the concentration of zinc multiplied by the control substance concentration;
administering a treatment for prostate cancer if the score is 85% or less of the average zinc score for a population of healthy adult males and not administering a treatment for prostate cancer if the score is greater than 85% of the average zinc score for a population of healthy adult males.

2. The method of claim 1, wherein the level of zinc is determined using ZPP1.

3. The method of claim 1, wherein the level of zinc is determined using a titration assay.

4. The method of claim 1, wherein the control substance comprises creatinine.

5. The method of claim 1, wherein the zinc score is determined by multiplying the level of zinc in uM by the level of creatinine in ug/dL.

6. The method of claim 1, wherein the treatment for prostate cancer is selected from the group consisting of:
radial prostatectomy; chemotherapy; temozolomide; hormone therapy; radiation therapy; external beam radiation therapy; brachytherapy; cryosurgery; high-intensity focused ultrasound; and surgery.

7. The method of claim 1, wherein, if the score is less than 85% and more than 55% of the average zinc score for a population of healthy adult males, the subject is administered further testing or monitoring for prostate cancer.

8. The method of claim 1, wherein a score of less than 85% of the average zinc score for a population of healthy adult males is equivalent to a zinc-creatinine score of 7 or less.

9. The method of claim 1, wherein a score of less than 55% of the average zinc score for a population of healthy adult males is equivalent to a zinc-creatine score of 4 or less.

10. An assay comprising:
contacting a series of solutions of detectable ZPP1 with portions of an expressed prostate secretion urine sample wherein the concentration of ZPP1 increases in a stepwise manner in the series of solutions;
detecting a signal from ZPP1 in each of the solutions;
determining, from the signal detected, the level of zinc in the sample;
determining the level of creatine in the sample;
determining a zinc score, wherein the zinc score is equal to the level of zinc in uM multiplied by the level of creatine in ug/dL.

11. The assay of claim 10, wherein the subject is determined to be at risk of prostate cancer if the score is 85% or less of the average zinc score for a population of healthy adult males and wherein the subject is determined to be at risk of having a high-risk prostate cancer if the zinc score is less than 55% of the average zinc score for a population of healthy adult males.

12. (canceled)

13. The assay of claim 10, wherein the zinc score of the subject is determined for a sample obtained at a first date and further comprising determining the zinc score in at least one additional sample obtained at a later date;
wherein a decrease in the zinc score over time indicates the subject is at risk of having or developing prostate cancer.

14. The assay of claim 10, wherein a treatment for prostate cancer is administered to a subject determined to be at risk of prostate cancer.

15. The assay of claim 14, wherein the treatment for prostate cancer is selected from the group consisting of:
radial prostatectomy; chemotherapy; temozolomide; hormone therapy; radiation therapy; external beam radiation therapy; brachytherapy; cryosurgery; high-intensity focused ultrasound; and surgery.

16. The assay of claim 10, wherein, if the score is greater than 85% of the average zinc score for a population of healthy adult males, the subject is recommended to not receive treatment for prostate cancer.

17. The assay of claim 10, wherein, if the score is less than 85% and more than 55% of the average zinc score for a population of healthy adult males, the subject is recommended to undergo further testing or monitoring for prostate cancer.

18. The assay of claim 10, further comprising the step of administering a further test or monitoring the subject for prostate cancer if the score is less than 85% and more than 55% of the average zinc score for a population of healthy adult males.

19. The assay of claim 10, wherein a score of less than 85% of the average zinc score for a population of healthy adult males is equivalent to a zinc-creatinine score of 7 or less.

20. The assay of claim 10, wherein a score of less than 55% of the average zinc score for a population of healthy adult males is equivalent to a zinc-creatine score of 4 or less.

21-68. (canceled)
